(19) United States
(12) Patent Application Publication
(10) Pub. No.: US 2017/0181661 A1
(43) Pub. Date: Jun. 29, 2017

(54) POSITION DETECTION SYSTEM AND GUIDANCE SYSTEM

(71) Applicant: OLYMPUS CORPORATION, Tokyo (JP)

(72) Inventor: Atsushi CHIBA, Tokyo (JP)

(73) Assignee: OLYMPUS CORPORATION, Tokyo (JP)

(21) Appl. No.: 15/459,095

(22) Filed: Mar. 15, 2017

Related U.S. Application Data
(63) Continuation of application No. PCT/JP2015/079869, filed on Oct. 22, 2015.

(30) Foreign Application Priority Data
Mar. 25, 2015 (JP) 2015-063040

(51) Int. Cl. A61B 5/06; A61B 1/00; A61B 5/07; A61B 1/04; A61B 5/00

(52) U.S. Cl. CPC A61B 5/062; A61B 1/041; A61B 5/742; A61B 5/073; A61B 1/00016; A61B 1/00025

(57) ABSTRACT

A position detection system includes a capsule medical device having therein a magnetic field generator configured to generate a magnetic field, magnetic field detectors configured to detect the magnetic field and output detection signals, and a processor including hardware. The processor is configured to: calculate a position of the capsule medical device by using at least one of the detection signals; determine whether the position of the capsule medical device falls within a predefined detection target region for the capsule medical device; determine whether proper position detection for the capsule medical device based on the detection signals is possible; and set a threshold to be used for determining whether the proper position detection for the capsule medical device is possible, based on the position of the capsule medical device if the position of the capsule medical device falls within the predefined detection target region for the capsule medical device.

### CROSS-REFERENCE TO RELATED APPLICATIONS

This application is a continuation of PCT International Application No. PCT/JP2015/079869 filed on Oct. 22, 2015 which claims the benefit of priority from Japanese Patent Application No. 2015-063040 filed on Mar. 25, 2015, the entire contents of which are incorporated herein by reference.

### BACKGROUND

1. Technical Field

The disclosure relates to a position detection system for detecting a position of a capsule medical device introduced into a subject. The disclosure also relates to a guidance system for guiding the capsule medical device.

2. Related Art

In the related art, medical devices have been developed which are configured to be introduced into a subject to acquire various kinds of information on an inside of the subject or to administer medicine and the like to the inside of the subject. As an example, there has been known capsule endoscopes small enough to be introduced into a digestive tract (inside lumen) of a subject. The capsule endoscope has a capsule-shaped casing inside which an imaging function and a radio communication function are provided, and also is to be swallowed by a subject and then performs imaging while moving inside a digestive tract, and sequentially performs radio transmission of image data of an image of an internal organ of the subject (hereinafter referred to as in-vivo image).

System for detecting a position of such a capsule medical device inside the subject have been developed. For example, in JP 2008-132047 A, disclosed is a position detection system in which a magnetic field generation coil configured to generate a magnetic field is provided inside a capsule medical device, the magnetic field generated from the magnetic field generation coil is detected by a sensing coil provided outside the subject, and position detecting calculation for the capsule medical device is performed based on intensity of the detected magnetic field.

The Detection accuracy of the capsule medical device introduced into the subject depends on an SN ratio of the magnetic field detected by the sensing coil and an arrangement condition of the sensing coil. Therefore, there is a need to arrange the sensing coil so as to minimize a position detection error for the capsule medical device even when the SN ratio is low.

### SUMMARY

In some embodiments, a position detection system includes a capsule medical device having therein a magnetic field generator configured to generate a magnetic field, a plurality of magnetic field detectors configured to detect the magnetic field generated by the magnetic field generator and output a plurality of detection signals, and a processor including hardware. The processor is configured to: calculate a position of the capsule medical device by using at least one of the plurality of detection signals respectively output by the plurality of magnetic field detectors; determine whether or not the position of the capsule medical device falls within a predefined detection target region for the capsule medical device; determine whether or not proper position detection for the capsule medical device based on the plurality of detection signals is possible; and set a threshold to be used for determining whether or not the proper position detection for the capsule medical device is possible, based on the position of the capsule medical device if the position of the capsule medical device falls within the predefined detection target region for the capsule medical device.

In some embodiments, a guidance system includes the position detection system including the capsule medical device further having a permanent magnet, a guidance magnetic field generator configured to generate a magnetic field to be applied to the permanent magnet, and a guidance magnetic field controller configured to control the guidance magnetic field generator to perform guidance control for changing at least one of a position and a posture of the capsule medical device.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an exemplary structure of a guidance system according to a first embodiment of the present invention;

FIG. 2 is a schematic diagram illustrating an exemplary internal structure of a capsule endoscope illustrated in FIG. 1;

FIG. 3 is a schematic diagram illustrating an exemplary structure of a guidance magnetic field generation device illustrated in FIG. 1;

FIG. 4 is a flowchart illustrating operation of the guidance system illustrated in FIG. 1;

FIG. 5 is a schematic diagram to explain a threshold setting method based on a position detection result;

FIG. 6 is a schematic diagram to explain a calculation method for an initial threshold (theoretical value);

FIG. 7 is a schematic diagram to explain a determining method by a noise determination unit;

FIG. 8 is a schematic diagram to explain a determining method by the noise determination unit;

FIG. 9 is a schematic diagram to explain a determination value decision method (4);

FIG. 10 is a schematic diagram to explain a determination value decision method according to a second embodiment of the present invention;

FIG. 11 is a schematic diagram to explain the determination value decision method according to the second embodiment of the present invention;

FIG. 12 is a schematic diagram to explain the determination value decision method according to the second embodiment of the present invention; and

FIG. 13 is a schematic diagram to explain a threshold setting method according to a third embodiment of the present invention.

### DETAILED DESCRIPTION

In the following, a position detection system and a guidance system according to embodiments of the present invention will be described with reference to the drawings. In the embodiments described below, a capsule endoscope orally introduced into a subject and configured to image the inside of the subject (inside of lumen) is exemplified as an aspect of a capsule medical device to be detected by the position detection system, but the present invention is not limited by the embodiments. In other words, the present invention can be applied to position detection for various kinds of medical devices formed in a capsule shape, for example, a capsule endoscope moving from an esophagus to an anus of a subject, a capsule medical device configured to deliver medicine and the like into the subject, and a capsule medical device including a pH sensor configured to measure pH inside the subject.

In the following description, each of the drawings is merely intended to schematically illustrate a shape, a size, and a positional relation to an extent such that the content of the present invention can be understood. Therefore, the present invention is not limited only by the shape, size, and positional relation exemplified in each of the drawings. The same reference signs are used to designate the same elements throughout the drawings.

### First Embodiment

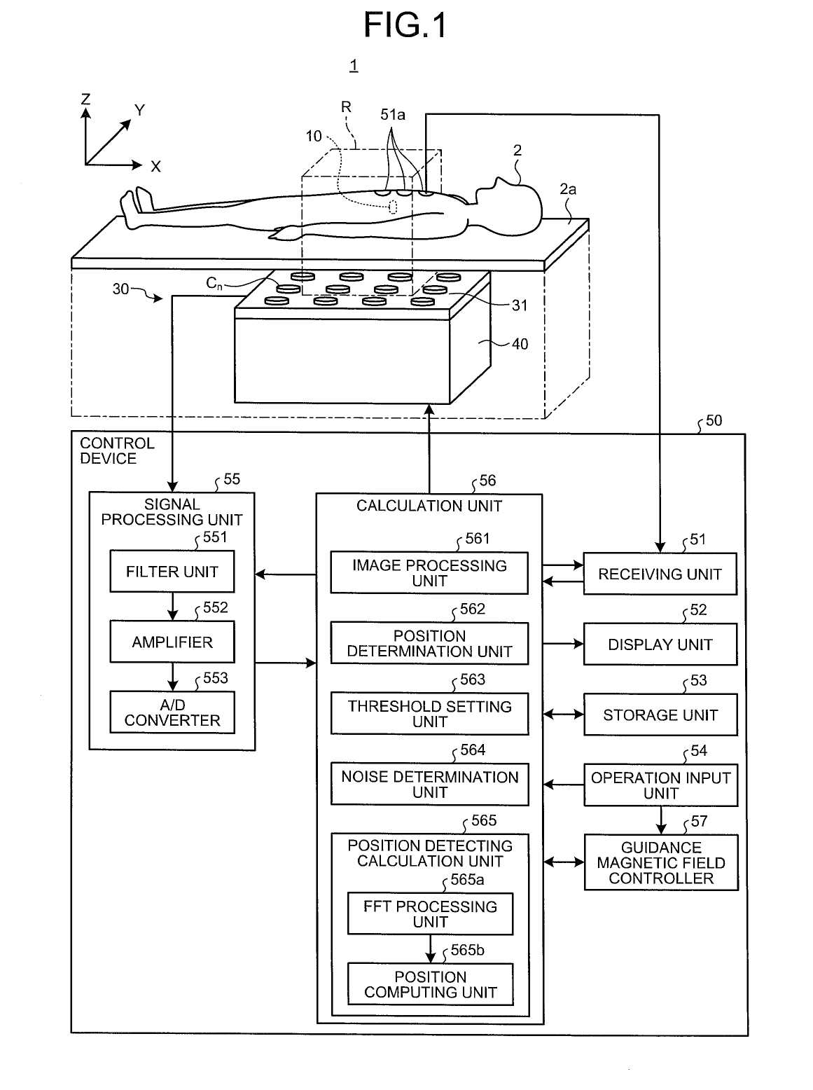

FIG. 1 is a schematic diagram illustrating an exemplary structure of a guidance system according to a first embodiment of the present invention. As illustrated in FIG. 1, a guidance system 1 according to the first embodiment includes: a capsule endoscope 10 as an example of a capsule medical device introduced into a lumen of a subject 2, configured to transmit image data obtained by imaging the inside of the subject 2 while superimposing the image data on a radio signal; a magnetic field detection device 30 provided below a bed 2a on which the subject 2 is placed, and configured to detect an alternating magnetic field generated by the capsule endoscope 10; a guidance magnetic field generation device 40 configured to generate a magnetic field to guide the capsule endoscope 10; and a control device 50 configured to detect a position of the capsule endoscope 10 based on the alternating magnetic field detected by the magnetic field detection device 30 and also guide the capsule endoscope 10 inside the subject 2.

In the following, an upper surface of the bed 2a, namely, a placement surface of the subject 2 is set as an XY surface (horizontal plane), and a direction orthogonal to the XY surface is set as a Z direction (vertical direction, namely, gravity direction).

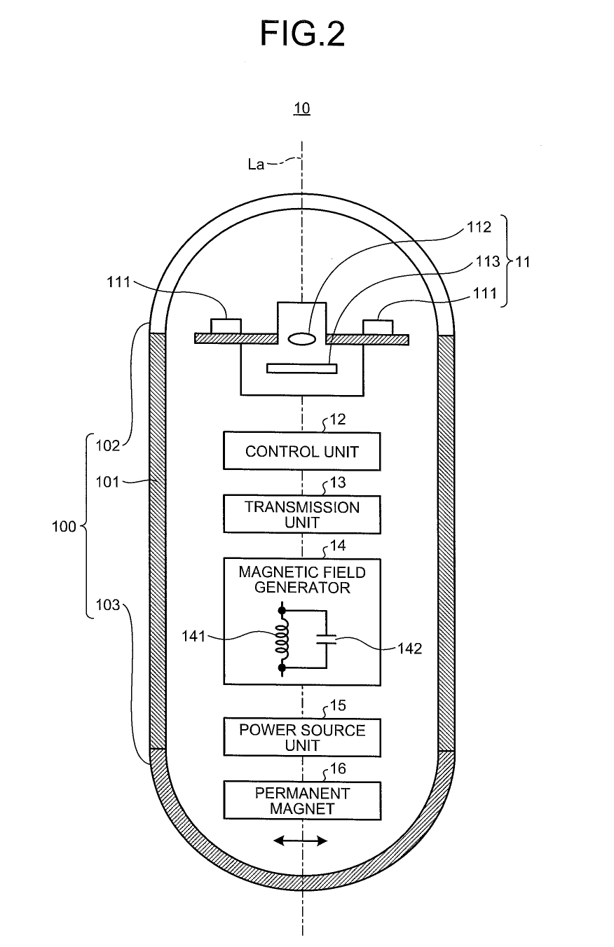

FIG. 2 is a schematic diagram illustrating an exemplary internal structure of the capsule endoscope 10 illustrated in FIG. 1. As illustrated in FIG. 2, the capsule endoscope 10 includes: a capsule-shaped casing 100 small enough to be easily introduced into the lumen of the subject 2; an imaging unit 11 housed inside the casing 100, configured to image the inside of the subject 2, and obtain an imaging signal; a control unit 12 configured to control operation of the respective units of the capsule endoscope 10 including the imaging unit 11 and also apply predetermined signal processing to the imaging signal obtained by the imaging unit 11; a transmission unit 13 configured to perform radio transmission for the imaging signal applied with the signal processing; a magnetic field generator 14 configured to generate an alternating magnetic field used for position detection for the capsule endoscope 10; a power source unit 15 configured to supply power to the respective units of the capsule endoscope 10; and a permanent magnet 16.

The casing 100 is an outer casing small enough to be introduced into an organ of the subject 2. The casing 100 includes a cylindrical casing 101 formed in a cylindrical shape and dome-shaped casings 102, 103 each formed in a dome shape, and is implemented by closing both opened ends of the cylindrical casing 101 with the dome-shaped casings 102, 103 each formed in the dome shape. The cylindrical casing 101 is formed of a colored member substantially opaque to visible light. At least one of the dome-shaped casings 102, 103 (in FIG. 2, dome-shaped casing 102 on the imaging unit 11 side) is formed of an optical member transparent to light in a predetermined wavelength band such as the visible light. In FIG. 2, one imaging unit 11 is provided only on the one dome-shaped casing 102 side, but two imaging units 11 may also be provided, and in this case, the dome-shaped casing 103 is also formed of the transparent optical member. The casing 100 liquid-tightly includes the imaging unit 11, control unit 12, transmission unit 13, magnetic field generator 14, power source unit 15, and permanent magnet 16.

The imaging unit 11 includes: an illumination unit 111 such as an LED; an optical system 112 such as a condenser lens; and an image sensor 113 such as a CMOS image sensor or a CCD. The illumination unit 111 emits illumination light such as white light to an imaging visual field of the image sensor 113 and illuminates the subject 2 inside the imaging visual field through the dome-shaped casing 102. The optical system 112 collects reflection light from the imaging visual field on an imaging surface of the image sensor 113 and forms an image. The image sensor 113 converts, to an electric signal, the reflection light (optical signal) from the imaging visual field received on the imaging surface, and outputs the same as an image signal.

The control unit 12 actuates the imaging unit 11 at a predetermined imaging frame rate and also makes the illumination unit 111 emit light in synchronization with the imaging frame rate. Furthermore, the control unit 12 generates image data by applying A/D conversion and other predetermined signal processing to the imaging signal generated by the imaging unit 11. The control unit 12 generates an alternating magnetic field from the magnetic field generator 14 by supplying power to the magnetic field generator 14 from the power source unit 15.

The transmission unit 13 includes a transmission antenna, acquires the image data on which the signal processing has been performed by the control unit 12 as well as acquires related information thereof, performs modulation processing on the acquired image data and related information, and sequentially transmits the same wirelessly to the outside via the transmission antenna.

The magnetic field generator 14 includes: a magnetic field generation coil 141 constituting a part of a resonance circuit and configured to generate a magnetic field when current flows; and a capacitor 142 configured to form the resonance circuit together with the magnetic field generation coil 141, and the magnetic field generator generates an alternating magnetic field having a predetermined frequency by receiving power supply from the power source unit 15.

The power source unit 15 is a power storage unit such as a button battery and a capacitor and includes a switch unit such as a magnetic switch or an optical switch. In the case of including the magnetic switch, the power source unit 15 switches on/off states of a power source by a magnetic field applied from the outside, and in the case of the on state, the power source unit 15 suitably supplies each unit (imaging unit 11, control unit 12, and transmission unit 13) of the capsule endoscope 10 with power of the power storage unit. Also, in the case of the off state, the power source unit 15 stops supplying power to the respective constituent units of the capsule endoscope 10.

The permanent magnet 16 is provided in order to enable magnetic guidance for the capsule endoscope 10 by the magnetic field generated by the guidance magnetic field generation device 40, and is fixed and arranged inside the capsule-shaped casing 100 such that a magnetization direction has an inclination angle relative to a long axis La of the casing 100. In FIG. 2, the magnetization direction of the permanent magnet 16 is indicated by arrows. In the first embodiment, the permanent magnet 16 is arranged in a manner such that the magnetization direction is orthogonal to the long axis La. The permanent magnet 16 operates by following a magnetic field applied from the outside, and as a result thereof, magnetic guidance for the capsule endoscope 10 by the guidance magnetic field generation device 40 can be implemented.

Referring again to FIG. 1, the magnetic field detection device 30 includes: a panel 31 having a flat surface; and a plurality of sensing coils Cₙ (n=1, 2, . . . ) disposed on a main surface of the panel 31 and each configured to receive an alternating magnetic field generated from the capsule endoscope 10 and output a detection signal. Each of the sensing coils Cₙ is a magnetic field detector formed of a cylindrical coil obtained by winding a coil wire member like a coil spring, and has an opening diameter of about 30 to 40 mm and a height of about 5 mm, for example.

The magnetic field detection device 30 thus structured is disposed in the vicinity of the subject 2 under examination. In the first embodiment, the magnetic field detection device 30 is disposed below the bed 2a in a manner such that the main surface of the panel 31 becomes horizontal.

A region where a position of the capsule endoscope 10 can be detected by the magnetic field detection device 30 is a detection target region R. The detection target region R is a three-dimensional closed region including a range inside the subject 2 where the capsule endoscope 10 is movable (namely, range of an organ to be observed), and is also preliminarily set in accordance with arrangement of the plurality of sensing coils Cₙ in the magnetic field detection device 30, intensity of a magnetic field that can be generated by the magnetic field generator 14 inside the capsule endoscope 10, and the like.

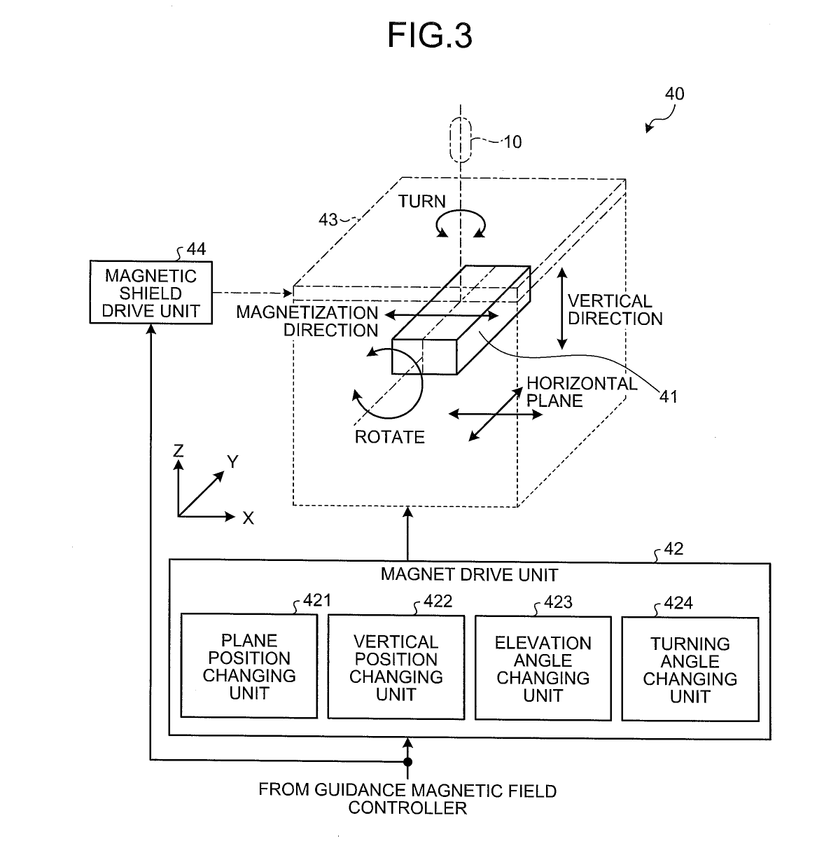

FIG. 3 is a schematic diagram illustrating an exemplary structure of the guidance magnetic field generation device 40. As illustrated in FIG. 3, the guidance magnetic field generation device 40 generates a magnetic field in order to relatively change a position of the capsule endoscope 10 introduced into the subject 2, an inclination angle and an orientation angle of the long axis La with respect to the vertical direction relative to the subject 2. More specifically, the guidance magnetic field generation device 40 includes: an extracorporeal permanent magnet 41 as the guidance magnetic field generator (second magnetic field generator) configured to generate a magnetic field; a magnet drive unit 42 configured to change a position and a posture of the extracorporeal permanent magnet 41; and a magnetic shield 43 and a magnetic shield drive unit 44, both of which function as a shield device for shielding the magnetic field generated by the extracorporeal permanent magnet 41. The magnet drive unit 42 includes a plane position changing unit 421, a vertical position changing unit 422, an elevation angle changing unit 423, and a turning angle changing unit 424.

The extracorporeal permanent magnet 41 is preferably implemented by a bar magnet having a cuboid shape, and restrains the capsule endoscope 10 within a region where one surface oriented parallel to the own magnetization direction among four surfaces is projected on a horizontal plane. An electromagnet for generating a magnetic field when current flows may also be provided instead of the extracorporeal permanent magnet 41.

The magnet drive unit 42 operates in accordance with a control signal output from a guidance magnetic field controller 57 described later. Specifically, the plane position changing unit 421 translates the extracorporeal permanent magnet 41 on the XY surface. In other words, the extracorporeal permanent magnet moves on the horizontal plane while a relative position between two magnetic poles magnetized in the extracorporeal permanent magnet 41 is secured.

The vertical position changing unit 422 translates the extracorporeal permanent magnet 41 in the Z direction. In other words, the extracorporeal permanent magnet moves in the vertical direction while the relative position between the two magnetic poles magnetized in the extracorporeal permanent magnet 41 is secured.

The elevation angle changing unit 423 changes an angle of the magnetization direction relative to the horizontal plane by rotating the extracorporeal permanent magnet 41 inside the vertical surface including the magnetization direction of the extracorporeal permanent magnet 41.

The turning angle changing unit 424 causes the extracorporeal permanent magnet 41 to turn relative to an axis in the vertical direction passing through a center of the extracorporeal permanent magnet 41.

The magnetic shield 43 is a plate-shaped member formed of ferromagnetic materials such as iron and nickel, and at least provided in an insertable and removable manner above the extracorporeal permanent magnet 41. The magnetic shield drive unit 44 inserts and removes the magnetic shield 43 in accordance with a control signal output from the guidance magnetic field controller 57 described later. While the magnetic shield 43 is removed from above the extracorporeal permanent magnet 41, a magnetic field is generated by the extracorporeal permanent magnet 41 in a space including the detection target region R. During this time, guidance for the capsule endoscope 10 by the guidance magnetic field generation device 40 can be performed. On the other hand, while the magnetic shield 43 is inserted into above the extracorporeal permanent magnet 41, the magnetic field generated by the extracorporeal permanent magnet 41 is shielded within the guidance magnetic field generation device 40. In other words, guidance for the capsule endoscope 10 is not performed during this time.

In the case of providing an electromagnet instead of the extracorporeal permanent magnet 41, the magnetic shield 43 and the magnetic shield drive unit 44 are not needed to be provided. In this case, magnetic field generation from the guidance magnetic field generation device 40 is stopped by stopping power supply to the electromagnet. Therefore, a power control unit that controls power supply to the electromagnet functions as a shield device of the magnetic field.

Referring again to FIG. 1, the control device 50 includes: a receiving unit 51 configured to receive a radio signal transmitted from the capsule endoscope 10 via a receiving antenna 51a; a display unit 52 configured to output and display, on a display device and the like, various kinds of information and the like processed by the control device 50; a storage unit 53; an operation input unit 54 used to input various kinds of information and a command for the control device 50; a signal processing unit 55 configured to generate magnetic field information by applying various kinds of signal processing to a detection signal output from each of the sensing coils Cₙ; a calculation unit 56 configured to perform various kinds of arithmetic processing such as image generation based on image data received by the receiving unit 51 and position detection for the capsule endoscope 10 based on the magnetic field information generated by the signal processing unit 55; and a guidance magnetic field controller 57 configured to perform control in order to guide the capsule endoscope 10.

At the time of performing the examination by the capsule endoscope 10, a plurality of receiving antennas 51a configured to receive radio signals transmitted from the capsule endoscope 10 is pasted on a body surface of the subject 2. The receiving unit 51 obtains image data and related information of an in-vivo image by selecting, from among these receiving antennas 51a, a receiving antenna 51a having highest reception intensity relative to a radio signal, and applying demodulation processing and the like to the radio signal received via the selected receiving antenna 51a.

The display unit 52 includes a various kinds of displays such as a liquid crystal and an organic EL, and displays, on a screen, various kinds of information received from the operation input unit 54, an in-vivo image of the subject 2, positional information of the capsule endoscope 10 at the time of imaging the in-vivo image, and the like.

The storage unit 53 is implemented by a storage medium and a writing reading unit that saves information in a rewritable manner, such as a flash memory or a hard disk. The storage unit 53 stores: various kinds of programs and various kinds of parameters for the calculation unit 56 to control the respective units of the control device 50; image data of an in-vivo image imaged by the capsule endoscope 10; positional information of the capsule endoscope 10 inside the subject 2; and the like.

The operation input unit 54 is implemented by various kinds of buttons, input devices such as a switch and a keyboard, pointing devices such as a mouse and a touch panel, a joystick, and the like, and feeds various kinds of information to the calculation unit 56 in accordance with input operation by a user. As the information received from the operation input unit 54, information to guide the capsule endoscope 10 to a position and a posture desired by the user (hereinafter referred to as guidance operational information) may be exemplified.

The signal processing unit 55 includes a filter unit 551 configured to shape a waveform of a detection signal output from the magnetic field detection device 30, an amplifier 552, and an A/D converter 553 configured to apply A/D conversion processing to the detection signal. In a space where the magnetic field detection device 30 can detect a magnetic field, there are an alternating magnetic field generated by the magnetic field generator 14 inside the capsule endoscope 10 and a guidance magnetic field formed by the guidance magnetic field generation device 40, but since these two magnetic fields have completely different frequencies, a problem of interference between the two magnetic fields hardly occurs.

The calculation unit 56 is formed by using, for example, a central processing unit (CPU) and the like and configured to read a program from the storage unit 53 and integrally control operation of the control device 50 by performing, for example, transference of a command and data to the respective units of the control device 50. The calculation unit 56 includes an image processing unit 561, a position determination unit 562, a threshold setting unit 563, a noise determination unit 564, and a position detecting calculation unit 565.

The image processing unit 561 generates image data for display by applying, to the image data received from the receiving unit 51, predetermined image processing such as white balance processing, demosaicing, gamma conversion, and smoothing (noise removal and the like).

The position determination unit 562 determines whether or not the position of the capsule endoscope 10 calculated by the position detecting calculation unit 565 falls within the detection target region R of the capsule endoscope 10.

The threshold setting unit 563 sets a threshold used for determination in the noise determination unit 564 based on a latest position detection result for the capsule endoscope 10.

The noise determination unit 564 determines whether to make the position detecting calculation unit 565 execute position detecting calculation for the capsule endoscope 10 based on an output value of a detection signal output from the signal processing unit 55 and the threshold set by the threshold setting unit 563.

If the noise determination unit 564 determines to execute the position detecting calculation, the position detecting calculation unit 565 obtains information (positional information) indicating a position of the capsule endoscope 10 based on the detection signal output from the signal processing unit 55. More specifically, the position detecting calculation unit 565 includes: an FFT processing unit 565a configured to extract amplitude, a phase, and the like of the alternating magnetic field by applying fast Fourier transform (hereinafter referred to as FFT processing) to detection data output from the signal processing unit 55; and a position computing unit 565b configured to calculate the position of the capsule endoscope 10 based on the magnetic field information extracted by the FFT processing unit 565a.

In the guidance system 1 illustrated in FIG. 1, the capsule endoscope 10, magnetic field detection device 30, signal processing unit 55, threshold setting unit 563, noise determination unit 564, and position detecting calculation unit 565 constitute the position detection system.

The guidance magnetic field controller 57 controls the respective units of the magnet drive unit 42 such that the capsule endoscope 10 takes a posture desired by a user at a position desired by the user based on the position and the posture of the capsule endoscope 10 calculated by the position detecting calculation unit 565 and guidance operational information received from the operation input unit 54. In other words, the capsule endoscope 10 is guided by changing a magnetic gradient in a space including the position of the capsule endoscope 10 by changing the position, elevation angle, turning angle of the extracorporeal permanent magnet 41.

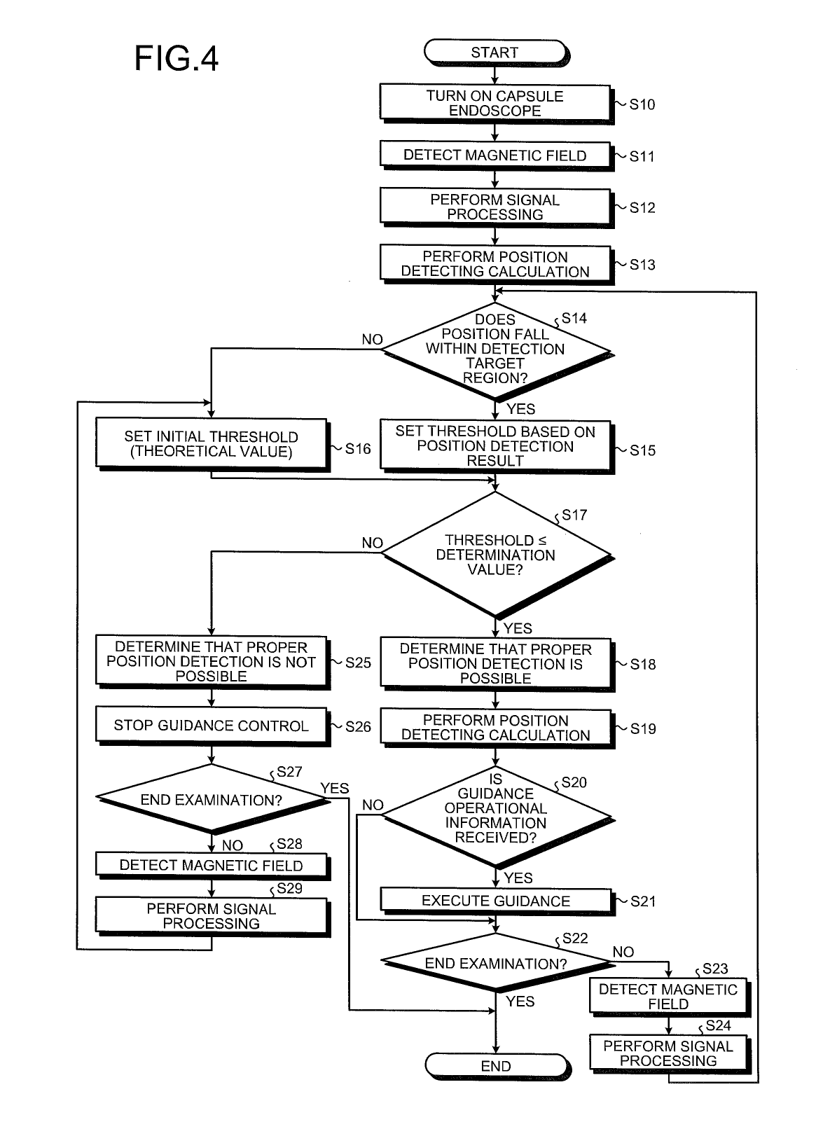

Next, operation of the guidance system 1 will be described. FIG. 4 is a flowchart illustrating operation of the guidance system 1.

First, in Step S10, a power source of the capsule endoscope 10 is turned ON. Consequently, power supply to the respective units of the capsule endoscope 10 is started from the power source unit 15 (see FIG. 2), the imaging unit 11 starts imaging, and also the magnetic field generator 14 starts generating a magnetic field.

In Step S11, the magnetic field detection device 30 detects the magnetic field. In other words, each of the sensing coil Cₙ of the magnetic field detection device 30 generates current in accordance with the magnetic field distributed in a position thereof, and outputs the current to the signal processing unit 55 as a detection signal of the magnetic field.

In Step S12, the signal processing unit 55 fetches a plurality of detection signals output from the magnetic field detection device 30 (current respectively generated from the plurality of sensing coils Cₙ), applies the signal processing such as waveform shaping, amplification, and A/D conversion to these detection signals, and outputs the same.

In Step S13, the position detecting calculation unit 565 performs position detecting calculation for the capsule endoscope 10 based on the plurality of detection signals output from the signal processing unit 55. More specifically, the FFT processing unit 565a applies the fast Fourier transform to each of the detection signals, thereby calculating amplitude and a phase of each of the detection signals. The amplitude and the phase correspond to intensity and a phase of the magnetic field at a position of each of the sensing coils Cₙ. The position computing unit 565b calculates a position and a posture of the capsule endoscope 10 based on the amplitude and the phase of the detection signal.

In subsequent Step S14, the position determination unit 562 determines whether the position of the capsule endoscope 10 calculated in Step S13 falls within the detection target region R of the capsule endoscope 10.

When the position of the capsule endoscope 10 falls within the detection target region R (Step S14: Yes), the threshold setting unit 563 sets a threshold to be used by the noise determination unit 564 based on the most recent result of the position detection of the capsule endoscope 10 (Step S15).

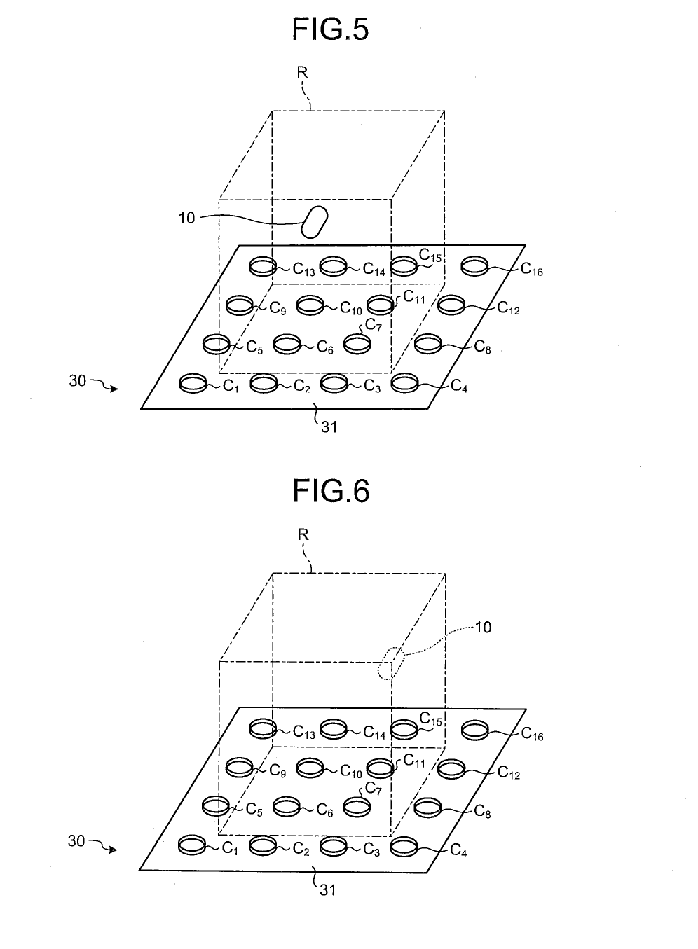

FIG. 5 is a schematic diagram to explain a threshold setting method based on a position detection result of the capsule endoscope 10, and illustrates a plurality of sensing coils Cₙ (as examples, n=1 to 16) disposed on the panel 31 of the magnetic field detection device 30 and the detection target region R of the capsule endoscope 10.

The threshold setting unit 563 obtains the most recent result of the position detecting calculation for the capsule endoscope 10 (Step S13 or Step S19 described later), and selects a sensing coil Cₙ predicted to have a maximal output value based on a positional relation between the result (namely, respective coordinate values of x, y, z of the capsule endoscope 10) and the plurality of sensing coils Cₙ. In other words, the sensing coil Cₙ located closest to the capsule endoscope 10 is selected. For example, if the capsule endoscope 10 exists at a position indicated in FIG. 5, a sensing coil C₁₀ is located closest to the capsule endoscope 10 and has the maximal output value. In this case, the threshold setting unit 563 sets, as a threshold, an output value of the sensing coil C₁₀ from among the detection signals output from the signal processing unit 55.

On the other hand, if the position of the capsule endoscope 10 does not fall within the detection target region R (Step S14: No), the threshold setting unit 563 sets, as a threshold to be used in the noise determination unit 564, an initial threshold (theoretical value) preliminarily stored (Step S16).

The initial threshold is preliminarily calculated based on output values (theoretical values) of the respective sensing coils Cₙ under the condition a detection level of each of the sensing coils Cₙ for the magnetic field generated by the capsule endoscope 10 becomes lowest. FIG. 6 is a schematic diagram to describe a calculation method for the initial threshold, and illustrates the plurality of sensing coils Cₙ disposed on the panel 31 of the magnetic field detection device 30 and the detection target region R of the capsule endoscope 10.

Suppose that the capsule endoscope 10 is disposed at a position among the detection target region R, in which the detection level of each of the sensing coils Cₙ for the magnetic field generated by the capsule endoscope 10 becomes lowest. Specifically, when the capsule endoscope 10 is located on an upper surface of the detection target region R, preferably, at an endmost portion of the upper surface, the detection level of each of the sensing coils Cₙ becomes lowest. In FIG. 6, illustrated is a case where the capsule endoscope 10 is located at one of four corners on the upper surface of the detection target region R.

In this case, an output value from a sensing coil Cₙ having a theoretically maximal output value is set as a threshold. In the case of FIG. 6, an output value of a sensing coil C₄ closest to the capsule endoscope 10 theoretically becomes maximal. Therefore, the output value of the sensing coil C₄ calculated based on intensity (theoretical value) of the magnetic field generated by the magnetic field generator 14 of the capsule endoscope 10 and a distance between the capsule endoscope 10 and the sensing coil C₄ at this point is set as an initial threshold.

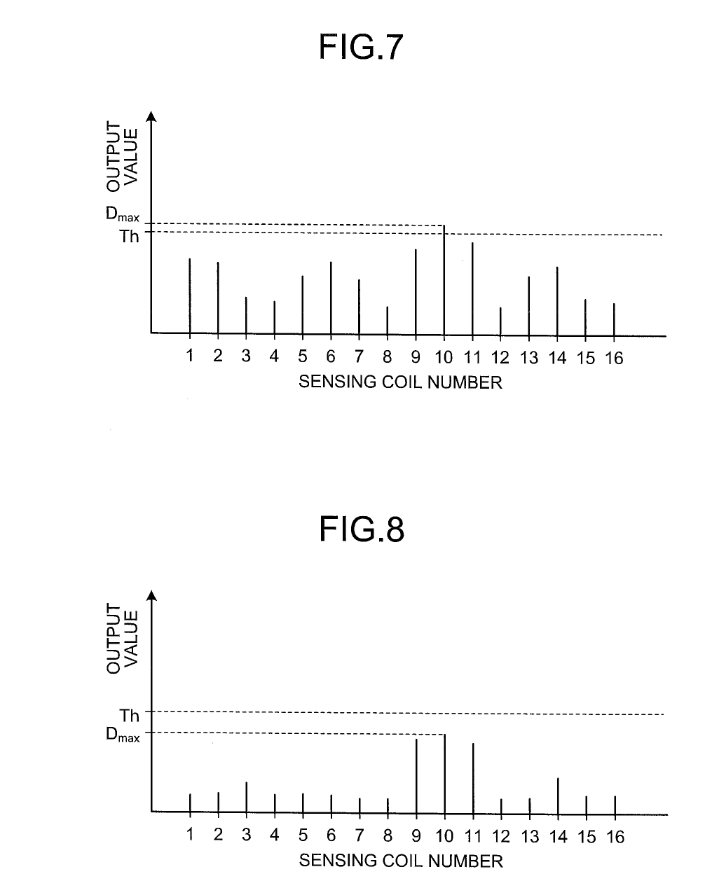

In Step S17, the noise determination unit 564 compares the threshold set by the threshold setting unit 563 in Step S15 or S16 with a determination value defined based on the output values (amplitude) of the plurality of detection signals output from the signal processing unit 55. A determination value decision method will be described later. FIGS. 7 and 8 are schematic diagrams to describe the determining method for an output value of a sensing coil. Here, as an example, a maximum value Dₘₐₓ among the output values of the plurality of sensing coils Cₙ is set as a determination value as illustrated in FIGS. 7 and 8, and the determination is made on whether this determination value Dₘₐₓ is equal to or more than a threshold Th.

As illustrated in FIG. 7, if the determination value (maximum value Dₘₐₓ) is equal to or more than the threshold Th (Step S17: Yes), the noise determination unit 564 determines that the capsule endoscope 10 is located inside the detection target region R and proper position detection is possible (Step S18). Here, “proper position detection is possible” means that a magnetic field component generated by the capsule endoscope 10 is contained in a signal detected by the sensing coil Cₙ and position detecting calculation can be performed based on this magnetic field component. In contrast, “proper position detection is not possible” means that not much magnetic field component generated by the capsule endoscope 10 is contained in the signal detected by the sensing coil Cₙ and position detecting calculation is executed based on a noise component.

In this case, the position detecting calculation unit 565 executes the position detecting calculation for the capsule endoscope 10 based on the plurality of detection signals output from the signal processing unit 55 (Step S19). Details of the position detecting calculation are the same as Step S13.

In subsequent Step S20, the guidance magnetic field controller 57 determines whether the guidance operational information is received from the operation input unit 54. If the guidance operational information is received (Step S20: Yes), the guidance magnetic field controller 57 executes guidance for the capsule endoscope 10 by controlling operation of the guidance magnetic field generation device 40 based on the position and the posture of the capsule endoscope 10 calculated in Step S19 (Step S21).

On the other hand, if no guidance operational information is received from the operation input unit 54 (Step S20: No), operation of the guidance system 1 directly proceeds to Step S22.

In Step S22, the control device 50 determines whether to end examination by the capsule endoscope 10. Specifically, if a predetermined time has passed after turning on the power source of the capsule endoscope 10 for which a command signal to end examination is received via the operation input unit 54, the control device 50 determines to end examination.

In ending the examination (Step S22: Yes), operation of the guidance system 1 ends. On the other hand, in the case of not ending examination (Step S22: No), the magnetic field detection device 30 detects a magnetic field generated by the capsule endoscope 10 and outputs current generated by each of the sensing coils Cₙ to the signal processing unit 55 as a detection signal of the magnetic field (Step S23).

In subsequent Step S24, the signal processing unit 55 fetches the plurality of detection signals output from the magnetic field detection device 30, applies the signal processing such as waveform shaping, amplification, and A/D conversion to these detection signals, and outputs the same. After that, operation of the guidance system 1 proceeds to Step S14.

On the other hand, in Step S17, as illustrated in FIG. 8, if the determination value (maximum value Dₘₐₓ) is less than the threshold Th (Step S17: No), the noise determination unit 564 determines that the capsule endoscope 10 is not located inside the detection target region R and the proper position detection is not possible (Step S25). In this case, operation of the position detecting calculation unit 565 shifts to Step S26 without performing position detecting calculation for the capsule endoscope 10.

In Step S26, the guidance magnetic field controller 57 stops guidance control for the capsule endoscope 10. Specifically, the guidance magnetic field controller 57 controls the magnetic shield drive unit 44 of the guidance magnetic field generation device 40 to shield the magnetic field generated by the extracorporeal permanent magnet 41 within the guidance magnetic field generation device 40 by inserting the magnetic shield 43 into above the extracorporeal permanent magnet 41. Consequently, the guidance magnetic field is not applied to the capsule endoscope 10 even though the guidance operational information is received from the operation input unit 54.

In subsequent Step S27, the control device 50 determines whether to end examination by the capsule endoscope 10. The determining method is the same as Step S22.

In ending the examination (Step S27: Yes), operation of the guidance system 1 ends. On the other hand, in the case of not ending the examination (Step S27: No), the magnetic field detection device 30 detects a magnetic field and outputs current generated by each of the sensing coils Cₙ to the signal processing unit 55 as a detection signal (Step S28).

In subsequent Step S29, the signal processing unit 55 fetches the plurality of detection signals output from the magnetic field detection device 30, applies the signal processing such as waveform shaping, amplification, and A/D conversion to these detection signals, and outputs the same. After that, operation of the guidance system 1 proceeds to Step S16. In other words, if it is determined that the proper position detection is not possible (Step S25), position detecting calculation is not performed. Therefore, in Step S16, an initial threshold (theoretical value) preliminarily calculated is set.

Next, the determination value decision method to be compared with the threshold in Step S17 will be described. As the determination value decision method, the following decision methods (1) to (4) may be exemplified. In Step S17 described above, a determination value defined by any one of the decision methods for the determination value (1) to (4) may be used.

Determination Value Decision Method (1)

As described in Step S17 above, a maximum value among output values of the plurality of sensing coils Cₙ is set as a determination value. For example, in the case of FIG. 7, since the output value of the sensing coil C₁₀ is maximal, this maxima value Dₘₐₓ is defined as a determination value and compared with the threshold Th.

Determination Value Decision Method (2)

An average value of a predetermined number (two or more) of output values in descending order among the output values of the plurality of sensing coils Cₙ is set as a determination value. For example, in the case of setting an average value of four output values in descending order as a determination value, when the output values illustrated in FIG. 7 are obtained, an average value of output values of the sensing coils C₁, C₉, C₁₀, and C₁₁ is defined as a determination value.

Here, an improper position (ghost) of the capsule endoscope 10 that can be detected by a position detection system in the related art depends on noise distribution. Therefore, a position and a signal level of the detected ghost are substantially constant. Therefore, by setting, as determination targets, the output values of the plurality of sensing coils Cₙ having a tendency to have large output values, it is possible to determine with high accuracy whether a current output value from each of the sensing coils Cₙ is a detection result of the magnetic field generated by the capsule endoscope 10 or a detection result of high-level noise.

Determination Value Decision Method (3)

Among the plurality of sensing coils Cₙ, output values of a sensing coil Cₙ having the maximal output value and at least one sensing coil Cₙ located in the vicinity of this sensing coil Cₙ are respectively set as determination values. For example, in the case of FIG. 7, since the output value of the sensing coil C₁₀ is maximal, the output value of the sensing coil C₁₀ and an output value of any one of the adjacent sensing coils C₆, C₉, C₁₁, and C₁₄ are respectively set as determination values (see FIG. 5). In this case, when the output value of the sensing coil C₁₀ and an output value of a sensing coil Cₙ adjacent thereto are equal to or more than the threshold Th, it is determined that the proper position detection is possible. Among the adjacent sensing coils C₆, C₉, C₁₁, and C₁₄, the sensing coil Cₙ from which a determination value is to be obtained may be preliminarily defined, or an output value of a sensing coil Cₙ located in a moving direction of the capsule endoscope 10 may also be used as a determination value.

Determination Value Decision Method (4)

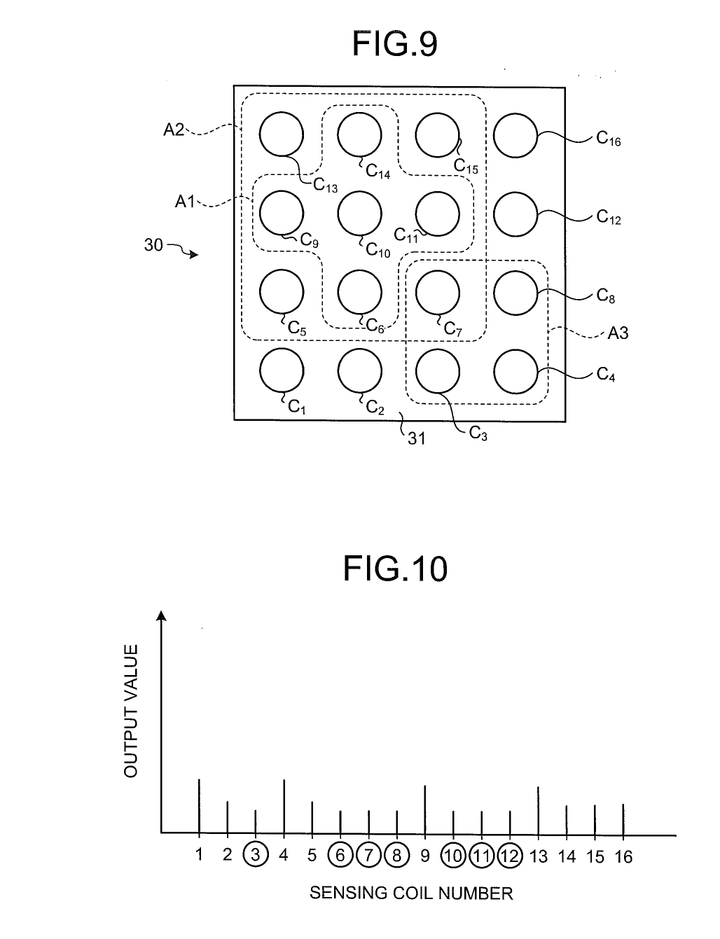

FIG. 9 is a schematic diagram to describe the determination value decision method (4) and also is an upper surface view illustrating the plurality of sensing coils Cₙ disposed on the panel 31. In the determination value decision method (4), among the plurality of sensing coils Cₙ, an average value of output values of a sensing coil Cₙ having a maximal output value and of a sensing coil Cₙ located in the vicinity of this sensing coil Cₙ is set as a determination value. For example, in the case of FIG. 7, since the output value of the sensing coil C₁₀ is maximal, an average value of the output values of the sensing coil C₁₀ and the sensing coils C₆, C₉, C₁₁, and C₁₄ located in the vicinity thereof is defined as a determination value as illustrated in FIG. 9, and compared with the threshold Th. As sensing coils Cₙ located in the vicinity, a group of adjacent sensing coils in a vertical direction and a lateral direction with respect to the sensing coil Cₙ having the maximal output value may be selected as illustrated in a region A1 of FIG. 9, or a group of adjacent sensing coils in the vertical direction, the horizontal direction, and an oblique direction may also be selected as illustrated in a region A2. Alternatively, if the sensing coil Cₙ having the maximal output value is located at an end portion of the panel 31 (e.g., sensing coil C₄), a group of sensing coils surrounding the sensing coil C₄ may be selected as illustrated in a region A3.

Here, when the capsule endoscope 10 is actually located inside the detection target region R and if there is a sensing coil Cₙ having a large output value, output values of sensing coils Cₙ surrounding the same have a tendency to be large, too. In contrast, if the capsule endoscope 10 is not located inside the detection target region R, even though there is a sensing coil Cₙ having a large output value, output values of sensing coils Cₙ located in the vicinity thereof do not constantly become large. Therefore, by comparing the average value of the output values of the sensing coil Cₙ having the maximal output value and of the sensing coils Cₙ in the vicinity thereof with the threshold Th, whether the capsule endoscope 10 exists inside the detection target region R can be accurately determined.

As described above, in the first embodiment of the present invention, the comparison is made between the threshold and the determination value defined based on the output value of the sensing coil Cₙ, and whether or not the proper position detection for the capsule endoscope 10 is possible is determined based on this comparison result. If it is determined that the proper position detection it not possible, the position detecting calculation unit 565 is inhibited from performing position detecting calculation. Therefore, a position detection result of the capsule endoscope 10 can be prevented from being improperly output.

Furthermore, according to the first embodiment of the present invention, the threshold to be compared with the determination value is updated every time the position of the capsule endoscope 10 is detected. Therefore, even if a noise level becomes higher than an assumed level or the noise level fluctuates, it is possible to accurately determine whether or not the proper position detection is possible. Therefore, even though a member that can be a noise generating source is used for a device constituting the guidance system 1 and a peripheral apparatus thereof, influence on a position detection result can be reduced.

Additionally, according to the first embodiment of the present invention, if the proper position detection for the capsule endoscope 10 is not possible, guidance control for the capsule endoscope 10 is stopped. Therefore, inappropriate guide based on an erroneously-detected position of the capsule endoscope 10 can be prevented.

### Modified Example 1-1

Next, a modified example 1-1 of the first embodiment of the present invention will be described.

In the determination value decision methods (2) and (4), the average value of the output values of the plurality of sensing coils Cₙ is defined as the determination value, but a sum of these output values may also be set as a determination value. In this case, it is preferable to adjust a threshold value to be used in Step S17 in accordance with the number of the output values used to define the determination value. For example, in the case of defining a sum of output values from five sensing coils Cₙ as a determination value, as for a threshold also, an output value of a sensing coil Cₙ predicted to have a maximal output value or a value five times of an initial value is set as the threshold.

### Modified Example 1-2

Next, a modified example 1-2 of the first embodiment of the present invention will be described.

In the first embodiment, the output value of the sensing coil Cₙ predicted to have the maximal output value is directly set as the threshold (see Step S15), but a temporal average value of this output value may also be set as a threshold. For example, if the sensing coil Cₙ predicted to have the maximal output value is the sensing coil C₁₀, the threshold setting unit 563 fetches output values of the sensing coil C₁₀ for a predetermined period, calculates a temporal average value of these output values, and sets this temporal average value as a threshold. Consequently, a threshold based on a position detection result is relaxed, and the probability of determining that the proper position detection is not possible can be reduced although a signal level of the magnetic field generated by the capsule endoscope 10 is high.

Alternatively, a value obtained by multiplying the output value of the sensing coil Cₙ having the maximal output value by a predetermined coefficient (e.g., 0.8 or more and less than 1) may also be set as a threshold. In this case also, the threshold value based on the position detection result can be relaxed.

### Second Embodiment

Next, a second embodiment of the present invention will be described. The second embodiment is similar to the first embodiment in configuration and operation of a guidance system (see FIGS. 1 and 4), but different in a determination value decision method to be compared with a threshold in Step S17.

In the first embodiment, output values are obtained from all of sensing coils Cₙ, and the determination value is defined based on these output values. However, a sensing coil Cₙ from which an output value is obtained at the time of defining a determination value may also be preliminarily selected at the time of calibration performed before starting examination by a capsule endoscope 10. In other words, a detection signal from each sensing coil Cₙ is obtained in a state in which no magnetic field is generated by the capsule endoscope 10 and a magnetic field generated by a magnetic field generator 14 does not influence a detection target region R, and a sensing coil Cₙ having a low noise level is preliminarily selected as a target sensing coil Cₙ from which a determination value is obtained. As a selecting method for a sensing coil Cₙ, up to predetermined number (one or more) of sensing coil(s) Cₙ may be selected in the order from a sensing coil having a lowest noise level, or all of the sensing coils Cₙ having a noise level of a predetermined value or less may also be selected. Therefore, all of the sensing coils Cₙ disposed on a panel 31 may be selected, and only one sensing coil Cₙ may be selected. In the latter case, an output value of the selected sensing coil Cₙ is directly used as a determination value.

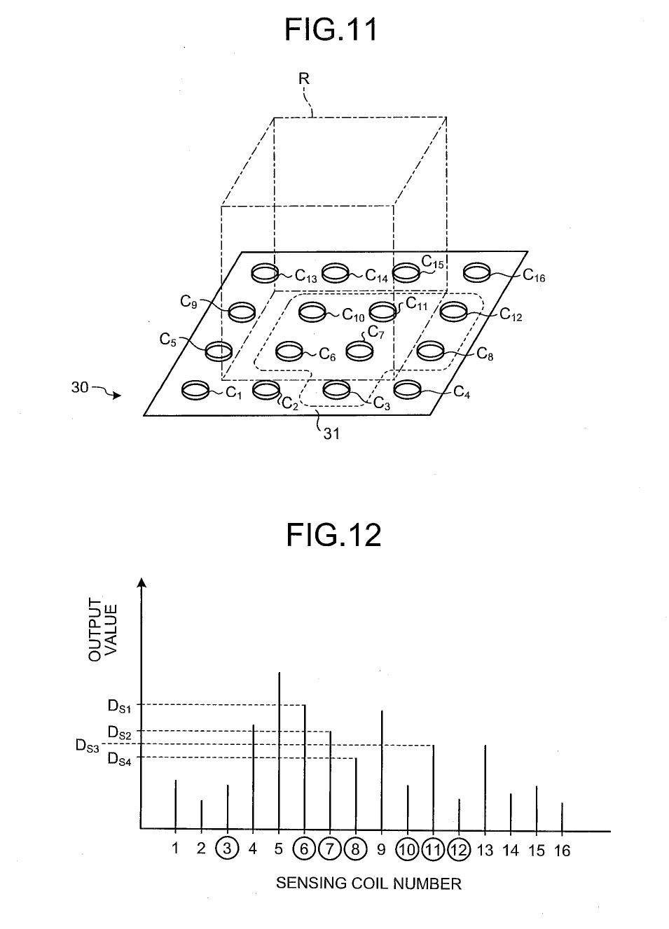

FIGS. 10 to 12 are schematic diagrams to explain a determination value decision method according to the second embodiment. For example, assume that output values (noise levels) of the sensing coils Cₙ as illustrated in FIG. 10 are obtained at the time of calibration before examination, and sensing coils C₃, C₆, C₇, C₈, C₁₀, C₁₁, and C₁₂ having low noise levels are selected as target sensing coils from which determination values are obtained (see FIG. 11). In Step S17, a determination value is defined based on the output values of these selected sensing coils C₃, C₆, C₇, C₈, C₁₀, C₁₁, and C₁₂. Numbers marked with circles in FIGS. 10 and 12 are coil numbers of the selected sensing coils Cₙ.

As an exemplary determination value decision method, a maximum value among the output values of the preliminarily selected sensing coils C₃, C₆, C₇, C₈, C₁₀, C₁₁, and C₁₂ is set as a determination value. For example, if output values of the respective sensing coils Cₙ illustrated in FIG. 12 are obtained after starting examination by the capsule endoscope 10, an output value D_{S1} of the sensing coil C₆ is maximal among the output values of the sensing coils C₃, C₆, C₇, C₈, C₁₀, C₁₁, and C₁₂. Therefore, this output value D_{S1} is defined as a determination value and compared with a threshold.

In preliminarily selecting sensing coils Cₙ by calibration, a threshold may also be set based on output values of the selected sensing coils Cₙ. In other words, in Step S15 of FIG. 4, an output value of a sensing coil Cₙ among the sensing coils Cₙ selected by calibration, which is located closest to the most recently detected position of the capsule endoscope 10, is set as a threshold.

### Modified Example 2-1

Next, a modified example 2-1 of the second embodiment of the present invention will be described.

As another exemplary determination value decision method, a predetermined number (two or more) of output values in descending order among the output values of the preliminarily selected sensing coils C₃, C₆, C₇, C₈, C₁₀, C₁₁, and C₁₂ may be selected as determination values. For example, if two output values in descending order are set as determination values, the output value D_{S1} of the sensing coil C₆ and an output value D_{S2} of the sensing coil C₇ are defined as determination values in FIG. 12. In this case, the output value D_{S1} and the output value D_{S2} are respectively compared with a threshold, and if both are equal to or more than the threshold, it is determined that the proper position detection for the capsule endoscope 10 is possible.

### Modified Example 2-2

Next, a modified example 2-2 of the second embodiment of the present invention will be described.

As still another exemplary determination value decision method, an average value of predetermined number (two or more) of output values in descending order among the output values of the preliminarily selected sensing coils C₃, C₆, C₇, C₈, C₁₀, C₁₁, and C₁₂ may be selected as a determination value. For example, in the case of setting an average value of four output values in descending order as a determination value, an average value of the output value D_{S1} of the sensing coil C₆, the output value D_{S2} of the sensing coil C₇, an output value D_{S3} of the sensing coil C₁₁, and an output value D_{S4} of the sensing coil C₈ is defined as a determination value in FIG. 12. Alternatively, a sum of the output values D_{S1}, D_{S2}, D_{S3}, D_{S4} may also be defined as a determination value.

### Modified Example 2-3

Next, a modified example 2-3 of the second embodiment of the present invention will be described.

As still another determination value decision method, an average value of output values of a sensing coil Cₙ having a maximal output value and sensing coils Cₙ located in the vicinity of this sensing coil Cₙ among sensing coils C₃, C₆, C₇, C₈, C₁₀, C₁₁, and C₁₂ preliminarily selected may be set as a determination value. For example, in FIG. 12, since the output value of the sensing coil C₆ is maximal among the sensing coils C₃, C₆, C₇, C₈, C₁₀, C₁₁, and C₁₂, an average value of output values of the sensing coil C₆ and the sensing coils C₃, C₇, and C₁₀ located in the vicinity thereof (see FIG. 11) is defined as the determination value. Alternatively, a sum of these output values may also be defined as a determination value.

### Third Embodiment

Next, a third embodiment of the present invention will be described. The third embodiment is similar to the first embodiment in configuration and operation of a guidance system (see FIGS. 1 and 4), but different in a threshold setting method based on a position detection result (see Step S15) used for determining whether or not the proper position detecting calculation is possible (see Step S17).

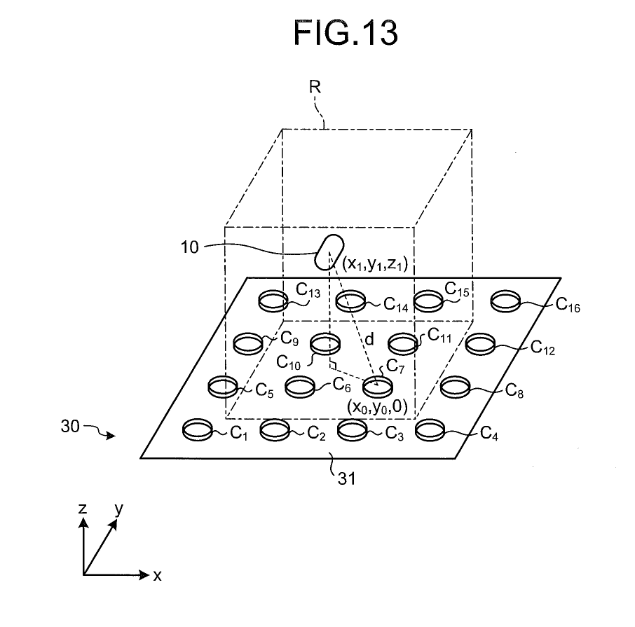

FIG. 13 is a schematic diagram to explain a threshold setting method based on a position detection result in the third embodiment. As a result of the most recently executed position detecting calculation (Step S13 or S19), if it is determined that a capsule endoscope 10 is located inside a detection target region R (Step S14: Yes), a threshold setting unit 563 sets a threshold based on a position and a posture of the capsule endoscope 10 (Step S15).

Specifically, the threshold setting unit 563 calculates a distance d between the capsule endoscope 10 and a preset specific sensing coil Cₙ. For example, if a position coordinate (x₁, y₁, z₁) of the capsule endoscope 10 is obtained and a sensing coil C₇ located at a coordinate (x₀, y₀, 0) is set as the specific sensing coil Cₙ, the distance d between the capsule endoscope 10 and the sensing coil C₇ is obtained by the following Formula (1).

d=√{(x₁−x₀)²+(y₁−y₀)²+z₁²} (1)

In FIG. 13, since an upper surface of a panel 31 is set as a reference surface, z-coordinates of all of the sensing coils Cₙ become zero.

The threshold setting unit 563 calculates intensity of a magnetic field at a position of the specific sensing coil C₇ based on the distance d and intensity of a magnetic field generated by a magnetic field generator 14 of the capsule endoscope 10. Alternatively, at this point, the intensity of the magnetic field may also be calculated considering the posture of the capsule endoscope 10. The threshold setting unit 563 sets the intensity of the magnetic field at the position of the specific sensing coil C₇ as a threshold.

In subsequent Step S17, as in the first embodiment, a maximum value of an output value of a sensing coil Cₙ, or an average value of the output values of the sensing coil Cₙ having the maximal output value and of the sensing coils Cₙ in the vicinity thereof is compared with the threshold (see determination value decision methods (1) to (4)). Alternatively, the output value of the specific sensing coil Cₙ used at the time of setting the threshold may also be set as a determination value. Alternatively, output values of the specific sensing coil Cₙ and sensing coils Cₙ adjacent thereto may also be respectively set as determination values, or an average value of the output values of the specific sensing coil Cₙ and of the sensing coils Cₙ located in the vicinity thereof may also be set as a determination value.

According to the third embodiment of the present invention, the magnetic field intensity (theoretical value) at the position of the specific sensing coil Cₙ calculated based on the most recently detected position of the capsule endoscope 10 is set as the threshold. Therefore, a detection signal (noise) having a level at which a ghost may be generated can be surely excluded without influence from noise level fluctuation. Therefore, detection of a ghost can be prevented.

### Modified Example 3

Next, a modified example 3 of the third embodiment will be described.

A ghost that can be detected by position detecting calculation tends to be generated in a region having a small z-coordinate, namely, in a region relatively close to a sensing coil Cₙ. Therefore, in the modified example 3, a threshold used for determining whether or not the proper position detection is possible (Step S15) is set based on a z-coordinate of the capsule endoscope 10 obtained by the most recently executed position detecting calculation, namely, based on a distance between the capsule endoscope 10 and the panel 31 where the sensing coils Cₙ is disposed.

More specifically, the z-coordinate (z=z₁) of a position coordinate (x₁, y₁, z₁) of the capsule endoscope 10 is obtained. Then, intensity of a magnetic field of each coil Cₙ if the z-coordinate of the capsule endoscope 10 is z₁ is calculated based on intensity of a magnetic field generated by the magnetic field generator 14 of the capsule endoscope 10. The threshold setting unit 563 sets a calculated value of this intensity as a threshold.

In subsequent Step S17, as in the first embodiment, a maximum value of an output value of a sensing coil Cₙ, or an average value of output values of the sensing coil Cₙ having the maximal output value and of the sensing coils Cₙ in the vicinity thereof is compared with the threshold (see determination value decision methods (1) to (4)).

### Fourth Embodiment

Next, a fourth embodiment of the present invention will be described.

In the first embodiment described above, if the proper position detection for a capsule endoscope 10 is not determined to be possible, a position detecting calculation unit 565 is inhibited from executing position detecting calculation, but the position detecting calculation unit may also be allowed to execute position detecting calculation. In this case, a calculation unit 56 may output information indicating that a position of the capsule endoscope 10 is erroneous and may make a display unit 52 display the information. Consequently, guidance operation for capsule endoscope 10 can be performed after a user recognizes the information displayed on the display unit 52 and indicating that the position of the capsule endoscope 10 is erroneous.

Alternatively, if it is determined that the proper position detection for the capsule endoscope 10 is not possible, the calculation unit 56 may stop display of a position of the capsule endoscope 10 on the display unit 52. Consequently, it is possible for a user to recognize that the proper position detection for the capsule endoscope 10 is not possible by finding a fact that display of the position of the capsule endoscope 10 is eliminated from the display unit 52.

### Fifth Embodiment

Next, a fifth embodiment of the present invention will be described.

In the first embodiment described above, if it is determined that the proper position detection for the capsule endoscope 10 is not possible, the guidance control for the capsule endoscope 10 is stopped. In contrast, when the proper position detection for the capsule endoscope 10 become possible, the guidance control may be started.

More specifically, in a guidance system 1, examination by the capsule endoscope 10 is started under the condition that a magnetic shield 43 of a guidance magnetic field generation device 40 is closed, namely, under the condition that the guidance control for the capsule endoscope 10 is not performed. Then, when a noise determination unit 564 determines that the proper position detection for the capsule endoscope 10 is possible (see Step S18 in FIG. 4), a guidance magnetic field controller 57 opens the magnetic shield 43. Consequently, a guidance magnetic field is generated in a space including a detection target region R, thereby achieving a state in which guidance control for the capsule endoscope 10 can be started.

In contrast, in the guidance system 1, the examination by the capsule endoscope 10 may also be started under the condition that the magnetic shield 43 of the guidance magnetic field generation device 40 is opened. In this case, when the noise determination unit 564 determines that the proper position detection for the capsule endoscope 10 is not possible (see Step S25 in FIG. 4), the guidance magnetic field controller 57 closes the magnetic shield 43. Consequently, the space including the detection target region R is shielded from a guidance magnetic field, thereby achieving a state in which guidance control for the capsule endoscope 10 cannot be started.

The first to fifth embodiments of the present invention and modified examples thereof are merely examples to implement the present invention, and the present invention is not limited thereto. Various kinds of inventions may be made by suitably combining a plurality of elements disclosed in the first and second embodiments and modified examples. The present invention can be modified in various ways in accordance with specifications and the like, and it is obvious from the above description that other various kinds of embodiments can be made within the scope of the present invention.

According to some embodiments, with reference to the threshold set based on the position of the capsule medical device calculated by the position detecting calculation unit, determination is made on whether or not the proper position detection for the capsule medical device is possible. With this feature, even if a noise level fluctuates, the determination can be made with high accurately, and if the capsule medical device is not located in the detection target region, it is possible to prevent the results of the position detection of the capsule medical device from being improperly output.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A position detection system comprising:
a capsule medical device having therein a magnetic field generator configured to generate a magnetic field;
a plurality of magnetic field detectors configured to detect the magnetic field generated by the magnetic field generator and output a plurality of detection signals; and
a processor comprising hardware, wherein the processor is configured to:
calculate a position of the capsule medical device by using at least one of the plurality of detection signals respectively output by the plurality of magnetic field detectors;
determine whether or not the position of the capsule medical device falls within a predefined detection target region for the capsule medical device;
determine whether or not proper position detection for the capsule medical device based on the plurality of detection signals is possible; and
set a threshold to be used for determining whether or not the proper position detection for the capsule medical device is possible, based on the position of the capsule medical device if the position of the capsule medical device falls within the predefined detection target region for the capsule medical device.

2. The position detection system according to claim 1, wherein the processor is configured to set a predetermined threshold if the position of the capsule medical device is outside the predefined detection target region.

3. The position detection system according to claim 1, wherein the processor is configured to set the threshold based on a relation between the position of the capsule medical device and positions of the plurality of magnetic field detectors.

4. The position detection system according to claim 3, wherein the processor is configured to set the threshold based on an output value of a magnetic field detector closest to the position of the capsule medical device, among the plurality of magnetic field detectors.

5. The position detection system according to claim 3, wherein the processor is configured to set the threshold based on a distance between the position of the capsule medical device and a position of a specific magnetic field detector of the plurality of magnetic field detectors.

6. The position detection system according to claim 3, wherein
the plurality of magnetic field detectors is disposed on a same plane, and
the processor is configured to set the threshold based on a distance between the position of the capsule medical device and the plane on which the plurality of magnetic field detectors is disposed.

7. The position detection system according to claim 1, wherein the processor is configured to:
set, as a determination value, a maximum value of output values of the plurality of detection signals; and
compare the determination value with the threshold to determine whether or not the proper position detection for the capsule medical device is possible.

8. The position detection system according to claim 1, wherein the processor is configured to:
decide a determination value by using a predetermined number of output values in descending order of output values of the plurality of detection signals; and
compare the determination value with the threshold to determine whether or not the proper position detection for the capsule medical device is possible.

9. The position detection system according to claim 1, wherein the processor is configured to:
decide a determination value by using a first output value of a first detection signal output by a first magnetic field detector of the plurality of magnetic field detectors and by using second output values of second detection signals output by a predetermined number of second magnetic field detectors of the plurality of magnetic field detectors, the first output value being a maximum value of output values of the plurality of detection signals, and the predetermined number of the second magnetic field detectors being adjacent to the first magnetic field detector; and
compare the determination value with the threshold to determine whether or not the proper position detection for the capsule medical device is possible.

10. The position detection system according to claim 7, wherein if the determination value is less than the threshold, the processor is configured to determine that the proper position detection for the capsule medical device is not possible.

11. The position detection system according to claim 1, wherein if the processor determines that the proper position detection for the capsule medical device is not possible, the processor does not calculate the position of the capsule medical device.

12. The position detection system according to claim 1, further comprising a display configured to display the position of the capsule medical device,
wherein, when the processor determines that the proper position detection for the capsule medical device is not possible, the display is configured to stop display of the position of the capsule medical device.

13. A guidance system, comprising:
the position detection system according to claim 1 comprising the capsule medical device further having a permanent magnet;
a guidance magnetic field generator configured to generate a magnetic field to be applied to the permanent magnet; and
a guidance magnetic field controller configured to control the guidance magnetic field generator to perform guidance control for changing at least one of a position and a posture of the capsule medical device.

14. The guidance system according to claim 13, further comprising a shield device configured to shield the magnetic field generated by the guidance magnetic field generator, and
when the processor determines that the proper position detection for the capsule medical device is not possible, the guidance magnetic field controller is configured to cause the shield device to shield the magnetic field generated by the guidance magnetic field generator.

15. The guidance system according to claim 13, wherein the guidance magnetic field controller is configured to switch between a state in which the guidance control is possible and a state in which the guidance control is not possible, depending on whether or not the processor determines that the proper position detection for the capsule medical device is possible.
